(19) United States
(12) Patent Application Publication
(10) Pub. No.: US 2011/0200986 A1
(43) Pub. Date: Aug. 18, 2011

(54) BIO-ASSAY USING LIQUID CRYSTALS

(75) Inventors: Kun-Lin Yang, Singapore (SG); Changying Xue, Singapore (SG); Saif A. Khan, Singapore (SG); Xinyan Bi, Singapore (SG)

(73) Assignee: NATIONAL UNIVERSITY OF SINGAPORE, (SG)

(21) Appl. No.: 13/122,947

(22) PCT Filed: Oct. 6, 2009
(86) PCT No.: PCT/SG2009/000365

§ 371 Date: Apr. 6, 2011

Related U.S. Application Data
(60) Provisional application No. 61/103,020, filed on Oct. 6, 2008.

(51) Int. Cl. G01N 33/543; C12Q 1/70; G01N 33/569; G01N 33/573; G01N 33/552; G01N 33/553; G01N 33/544

(52) U.S. Cl. 435/5; 436/518; 435/7.32; 435/7.4; 436/527; 436/525; 436/528; 422/68.1

(57) ABSTRACT

There is provided a method, an in vitro method and a detection system for detecting the presence of at least one biological molecule in at least one sample. The method can include, for example, providing a sample and/or a binding agent to a contact portion of a surface by way of at least one microfluidic channel; disposing a liquid crystal at the contact portion; determining whether the orientation of the liquid crystal changes after the sample contacts the binding agent, indicating the presence of the biological molecule; and determining length of bright region of the liquid crystal and/or change in interference color of said liquid crystal, and consequently indicating the quantity of said biological molecule. Also disclosed are methods of detecting biological molecules using at least one 4′-pentyl-biphenyl-4-R, where R may be at least one functional group selected from carboxylic acid, amine, aldehyde, and oligopeptide.

### FIELD OF THE INVENTION

The present invention relates to a method, an in vitro quantitative method and a detection system for detecting and/or quantifying the presence of at least one biological molecule in a sample. The invention further relates to a method of making the system.

### BACKGROUND OF THE ART

The application of microfluidics is revolutionizing the way activities are performed in a substantial proportion of chemical and biological operations. One use of microfluidics is in the manipulation of small volumes of liquids or liquid compositions on a solid substrate, where a network of channels and reservoirs are present for use in immunoassays. These microfluidic systems require less sample volume and have faster reaction times which make their use popular in immunoassays. However, the major challenge to miniaturize current microfluidic immunoassays into useful lab-on-chip devices is the detection mechanism. Since most microfluidic systems still heavily rely on enzyme catalyzed reactions (e.g. enzyme-linked immunosorbent assays, ELISA) or fluorescence (e.g. immunofluorescence assays) for detection, the use of bulky equipment such as spectrometers or fluorescent microscopes preclude the use of microfluidic immunoassays for point-of-care (POC) applications.

Furthermore, these methods of detection, involve labeling antibodies with enzymes or fluorescence probes which require additional working steps that not only further limit the possibility of preparing a fully integrated system but also slows down the process of detection and is not always accurate in detecting and quantifying small changes of the compound to be detected.

### SUMMARY OF THE INVENTION

The present invention addresses the problems above, and in particular provides a new, label-free detection and quantification mechanism based on microfluidics and interactions between liquid crystals (LCs) and antibodies. The present invention also provides a method of detection that by way of detecting small changes in at least one parameter, may be able to detect the presence of a compound of interest.

According to a first aspect, the present invention provides a quantitative method of detecting and/or quantifying the presence of at least one biological molecule in at least one sample, the method comprising the steps of:
(a) contacting a substrate with the at least one sample at a contact portion of said substrate;
(b) providing at least one binding agent specific to the biological molecule on said contact portion;
(c) disposing a liquid crystal at said contact portion with the sample and binding agent; and
(d) determining whether the orientation of the liquid crystal changes after the sample contacts the binding agent, indicating the presence of the biological molecule;
(e) determining length of bright region of the liquid crystal and/or change in interference colour of said liquid crystal, and consequently indicating the quantity of said biological molecule;
   wherein the sample and/or binding agent are provided to the contact portion of the substrate by way of at least one microfluidic channel.

Whilst the term “biological molecule” is described, it will be appreciated that the system and method according to the present invention is applicable to other forms of molecules, for example, but not limited to organic molecules.

For convenience, the term “antibodies” has been referred to as a target for the detection and quantification aspects of the invention. It will be appreciated that this is a convenient description, and that the invention is directed to detection and quantification of a variety of molecules within the sample, and not limited to antibodies.

The sample may include any particles, chemicals, elements, cells, specimen or culture obtained from any source, including without limitation chemical, biological and environmental samples. The sample may be obtained from animals (including humans) and encompass fluids, solids, tissues, and gases. Biological samples include, without limitation, cells and any components thereof, blood products, such as plasma, serum and the like, proteins, peptides, amino acids, polynucleotide, lipids, carbohydrates, and any combinations thereof.

In one embodiment, step (a) may be carried out before step (b) or step (b) may be carried out before step (a). Further, the sample may be first immobilised on the contact portion of the substrate by applying the sample to the contact portion of the substrate before the binding agent may be provided to the contact portion of the substrate by way of at least one microfluidic channel. Alternatively, the binding agent may be first immobilised on the contact portion of the substrate by applying the binding agent to the contact portion of the substrate before the sample may be provided to the contact portion of the substrate by way of at least one microfluidic channel. Further still, the sample and the binding agent may each be provided to the contact portion of the substrate separately by way of different microfluidic channels.

The use of microfluidic channels may make the analysis of the sample rapid and convenient due to the fast mass transport and the potential for integration into a single lab-on-chip device. The microfluidic channels may also be suitable for use with a very small sample volume, which is especially important when human samples may be used.

In one embodiment, the sample may contact the contact portion of the substrate by way of microfluidic channels running perpendicular to microfluidic channels providing the binding agent specific to the biological molecule on to the contact portion of the substrate. This arrangement may ensure that binding only takes place on the contact portion of the substrate where both the sample and binding agent come in contact with each other and not in the microfluidic channels.

Further, the method of the present invention may further comprise a step of removing the microfluidic channel from the substrate after steps (a) and/or (b). The microfluidic channel may be removed after it has provided the sample and/or binding agent to the contact portion of the substrate. The microfluidic channel may be removed prior to the step of disposing a liquid crystal at the contact portion with the sample and binding agent.

The microfluidic channels may be formed within at least one hydrophilic layer. The hydrophilic layer may minimize or inhibit protein binding. As expected binding may only take place on the contact portion of the substrate between the binding agent and the sample, the microfluidic channels inhibiting protein binding on its surface may be beneficial.

Some examples of hydrophilic layer may include a coated surface, a gel matrix, a polymer, a sol-gel monolith and a chemically modified surface. Coatings, for example, may include but are not limited to cellulose polymer, polyacrylamide, polydimethylacrylamide, acrylamide-based copolymer, polyvinyl alcohol, polyvinylpyrrolidone, polyethylene oxide, Pluronic™ polymers or poly-N-hydroxyethylacrylamide, Tween™ (polyoxyethylene derivative of sorbitan esters), dextran, a sugar, hydroxyethyl methacrylene, and indoleactic acid. A variety of methods are known to modify surfaces to make them hydrophilic (see e.g., Doherty et al, Electrophoresis, vol. 24, pp. 34 54, 2003). For example, an initial derivatization, often using a silane reagent, may be followed by a covalently bound coating of a polyacrylamide layer. This layer can be either polymerized in-situ, or preformed polymers may be bound to the surface. Examples of hydrophilic polymers that have been attached to a surface in this way include polyacrylamide, polyvinylpyrrolidone, and polyethylene oxide. Another method of attaching a polymer to the surface is thermal immobilization, which has been demonstrated with polyvinyl alcohol. In many cases, it may be sufficient to physically adsorb a polymeric coating to the surface, which has been demonstrated with cellulose polymers, polyacrylamide, polydimethylacrylamide, polyvinyl alcohol, polyvinylpyrrolidone, polyethylene oxide, Pluronic™ polymers (PEO-PPO-PEO triblock copolymers), and poly-N-hydroxyethylacrylamide. Certain techniques of surface modification are specific to polymer surfaces, for instance alkaline hydrolysis, or low-power laser ablation.

The hydrophilic layer may be completely made from Polydimethylsiloxane (PDMS) and/or Poly(methyl methacrylate) (PMMA). PDMS which is particularly known for its unusual rheological (or flow) properties and which is optically clear, and is generally considered to be inert, non-toxic and non-flammable is especially suitable for making the hydrophilic layer. PMMA a thermoplastic which is transparent is easy to handle and process at low cost.

As used herein, the term “substrate” is to refer to solid planar substrates having first and second opposing, or substantially parallel, planar surfaces. The substrate may be made from any one or a combination of polymer, ceramic, glass, metal, composite thereof and/or laminate thereof. This may especially be suitable for providing a contact surface for binding of the sample and/or the binding agent.

The substrate may be coated with N,N-dimethyl-n-octadecyl-3-aminopropyltrimethoxysilyl chloride (DMOAP), octadecyltrichlorosiliane or octyltrichlorosilane. DMOAP coated surfaces may adsorb proteins strongly and may align LCs homeotropically (perpendicular to the surface). Further, the substrate may be a DMOAP-coated glass slide which may provide homeotropic boundary conditions for LC alignment on the substrate. This kind of boundary conditions may lead to discontinuous orientation change of LC which may produce a clear and sharp responded optical image of the LC when added. LCs supported on DMOAP-coated glass slides may be used to build protein assays with extraordinary sensitivity and high reproducibility.

As used herein the term “biological molecule” refers to but is not limited to a nucleic acid, protein, antibody, carbohydrate, polysaccharide, lipid, and the like.

The biological molecule may be a macromolecule that has been shown to be or is believed to be functional. The biological molecule may at least be one of: DNA, RNA, protein, virus, bacteria, pathogen, antigen, or antibody. In one embodiment, the binding agent may be a compound that binds specifically to said biological molecule. The binding agent may be a protein, peptide, nucleic acid, small molecule, etc. Further, the binding agent may at least be one antibody.

As used herein, the term “nucleotide” may include, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide.

As used herein, the term “polynucleotide” refers to but is not limited to a nucleic acid sequence (such as a linear sequence) of any length.

The antibody may be any immunoglobulin, including antibodies and fragments thereof, that binds to a specific epitope. The antibody may be prepared against the biological molecule, a derivative and/or a fragment thereof. Such antibodies include, but are not limited to polyclonal, monoclonal, chimeric, humanised, single chain, Fab, Fab′, F(ab)′ fragments and/or F(v) portions of the whole antibody.

The contacting of the biological molecule and the binding agent may result in change in at least one parameter, the change may be detectable and/or quantifiable by the liquid crystal. In particular, the change in parameter may be at least one change in temperature, pH, concentration of H+ or other ions, concentration of at least one small molecule and the like.

In one embodiment, the liquid crystal may be a thermotropic, smectic, nematic, cholesteric or lyotropic LC. The LC may be a thermotropic LC. Further, the LC may be 4′-pentyl-biphenyl-4-R (where R is a functional group). The functional group may be carboxylic acid, amine, aldehyde, oligopeptide and the like. In particular, the LC may be 4-cyano-4′-pentylbiphenyl (5CB), 4′-pentyl-biphenyl-4-carboxylic acid (PBA) doped 5CB, 4,4′-biphenyldicarbonitrile, 4′-pentyl-biphenyl-4-carboxylic acid, 4′-pentyl-biphenyl-4-carboxamide and the like. The use of a label-free, LC based detection method greatly simplifies the analysis process, because orientations of LCs can respond to minute changes on solid surfaces and generate optical signals that may be visualized with the naked eye. There may also be no need for labelling target proteins with fluorescence dyes, which makes the direct analysis of the samples extracted from human bodies possible. Further, the concentration of the biological molecule may be determined quantitatively either through the interference color of LCs or through the length of bright LC region (with the naked eye under ambient conditions), due to the unique birefringent properties of LCs. A dark-to-bright transition of LCs occurs within a very small concentration range of biological molecules. Therefore, LCs may be used for quantifying the concentration of biological molecules. As LCs are optically birefringent, the distorted orientational profile may cause a particular interference colour under crossed polarized light. This characteristic of LC may also be used to determine concentration of the biological sample.

The use of microfluidic channels and LCs in combination in the method of detection of a biological molecule may thus provide a cost-effective method for rapid, sensitive and quantitative protein detection and analysis.

According to another aspect, the present invention provides an in vitro method for diagnosing a subject as having a disorder, wherein the method is according to that disclosed above, and wherein the presence of the biological molecule in the sample indicates the presence of the disorder. In one embodiment, the sample may be any tissue or fluid from a human, animal or plant. Further, the sample may be saliva, blood, blood plasma, urine and the like.

In one embodiment, the disorder may be any infectious disease. Further, the disease may be Dengue fever, AIDS, Hepatitis, sexual transmitted diseases, antibiotic resistance and the like. The in vitro method of the present invention may provide a medical diagnostic platform using microfluidic systems and liquid crystals (LCs) that may be used in clinical diagnosis of infectious diseases, or fast-screening of a suspected person who may carry Dengue fevers, AIDS, sexual transmitted diseases, or Hepatitis through airports or checkpoints.

According to another aspect, the present invention provides a detection system for carrying out a method and/or in vitro method as disclosed above, the detection system comprising:
a substrate and a hydrophilic layer with embossed microfluidic channels, engageable to form a path to transmit at least one sample and/or binding agent to a contact portion of the substrate via the microfluidic channels; wherein the substrate is removable to form a component of a liquid crystal cell for optical detection of bound biological molecule and binding agent on said contact portion.

This new type of diagnostic system provides a facile means of reporting assay results and may find useful applications in fast diagnosis of diseases, detecting pathogens, as a laboratory tool for research or diagnostic studies and in environmental monitoring. When used to describe a fluidic element, such as a passage, chamber or conduit, the terms “microfabricated” or “microfluidic” generally refer to one or more fluid passages, chambers or conduits which have at least one internal cross-sectional dimension, e.g., depth, width, length, diameter, etc., that is less than about 500 μm, and typically between 0.1 μm and 500 μm. The microfluidic channel has at least one cross-sectional dimension between 0.1 μm and 200 μm, between 0.1 μm and 100 μm, or between 1 μm and 20 μm. Accordingly, the detection systems prepared in accordance with the present invention typically include at least one microfluidic channel, usually at least two parallel microfluidic channels, and often, three or more parallel channels disposed within a single body structure. Channel intersections may exist in a number of formats, including cross intersections, “T” intersections, or any number of other structures whereby two channels are in fluid communication.

The term “channel” as used herein, refers to a microfluidic channel and describes fluid elements dimensioned so that flow therein is substantially laminar.

Optical detection of bound biological molecule and binding agent may be visualized with the naked eye. Alternatively, the optical textures of the LCs inside the LC cell may be observed under a polarized microscope.

In one embodiment, the LC cell comprises the removable substrate and a transparent cover over the substrate. The transparent cover may be any sheet of a solid substrate, flexible or otherwise which is transparent. The transparent cover may be a glass slide. The LC cell may further comprise a space between the transparent cover and the removable substrate. The space may be a fixed distance or it may be variable. The distance may be between the range of 1 μm to 100 μm. The distance may be between the range of 5 μm to 8 μm. Further, the distance may be about 6 μm.

The detection system may be used in quantitative protein detection.

As used herein, the term “microfabricated” generally refers to structural elements or features of a device that have at least one fabricated dimension in the range of from about 0.1 μm to about 500 μm. Thus, a device referred to as being microfabricated will include at least one structural element or feature having such a dimension.

The substrate may be bound to the hydrophilic layer. Pressure may be applied to ensure good sealing between the microfluidic channels and the substrate of the detection system.

According to another aspect, the present invention provides a method of quantifying and/or detecting the presence of at least one biological molecule in at least one sample, the method comprising the step of determining length of bright region of liquid crystal in contact with said sample and/or change in interference colour of said liquid crystal in contact with said sample, consequently indicating the quantity of said biological molecule in said sample.

According to another aspect, the present invention provides a detection system for detecting the presence of at least one biological molecule in at least one sample, the detection system comprising:
a substrate and a hydrophilic layer with embossed microfluidic channels, engageable to form a path to transmit the at least one sample and/or binding agent to a contact portion of the substrate via the microfluidic channels; a liquid crystal cell mounting for receiving the substrate to form a liquid crystal cell;
   wherein the substrate is removable from the hydrophilic layer and mountable to the liquid crystal cell mounting for optical detection of bound biological molecule and binding agent on said contact portion.

In one embodiment, the LC cell mounting may comprise a means of mounting the substrate comprising the biological molecule and binding agent on to the LC cell. Further, the mounting means may comprise a transparent cover to protect the contact portion of the substrate. The transparent cover may be kept away from interacting with the contact portion of the substrate by way of separators that may be arranged to surround the contact portion. The transparent cover may be a circular, oval or annular in shape. Further, the transparent cover is a glass slide. The separators may be placed at two sides of the glass slide to keep the cover from interacting with the contact portion of the substrate. The LC cell mounting may further comprise a support base for holding the substrate.

According to another aspect, the present invention provides a method of detecting a biological molecule in at least one sample using at least one 4′-pentyl-biphenyl-4-R (where R is a functional group). R may be carboxylic acid, amine, aldehyde, oligopeptide and the like. In particular, the 4′-pentyl-biphenyl-4-R may be 4-cyano-4′-pentylbiphenyl (5CB), 4′-pentyl-biphenyl-4-carboxylic acid (PBA) doped 5CB, 4,4′-biphenyldicarbonitrile, 4′-pentyl-biphenyl-4-carboxylic acid, 4′-pentyl-biphenyl-4-carboxamide and the like.

More in particular, the 4′-pentyl-biphenyl-4-R may be capable of detecting at least one change in at least one parameter resulting from contacting at least one binding agent specific to the biological molecule with the sample.

The parameter may be at least temperature, pH, concentration of H+ or ions and concentration of at least one small molecule, enzyme substrate and the like.

In one embodiment, the 4′-pentyl-biphenyl-4-R may be at least one PBA-doped 5CB, and the parameter may be a change in pH. Using 5CB doped with PBA, which has a pH sensitive functional group and a similar structure with 5CB, allows detection of small pH changes with a fast response time. As pH of the aqueous solution changes, orientations of LC undergo a homeotropic-to-planar or planar-to-homeotropic transition which can be easily visualized as an optical dark or bright image. The pH-driven optical response may be attributed to the protonation and deprotonation of PBA at the aqueous/LC interface, which induces the orientational transitions of 5CB.

In one embodiment, the method may be used to detect a biological molecule that results in a change in pH when the biological molecule comes in contact with at least one binding agent specific to the biological molecule in the sample.

The change in pH may be at least 0.01 to 7. More in particular, the change in pH may be 0.03 to 6.8, 0.05 to 6.5, 0.08 to 6.2, 0.1 to 6, 0.15 to 5.5, 0.2 to 5, 0.5 to 4 and the like.

The biological molecule may be at least one enzyme. More in particular, the enzyme may be phospholipases, cellulase, alcohol dehydrogenase and/or at least one beta-lactamase. The beta-lactamase may be penicillinase, cephalosporinase, carbenicillinase, cloxacilanase, oxacillinase, carbapenamase, metalloenzyme and the like.

The binding agent may be at least one substrate or antibiotic. The antibiotic may be at least one β-lactam antibiotic. The β-lactam antibiotic may be penicillin, cephalosporin, cephamycin, carbapenem (ertapenem) and the like.

The method of the present invention may be may be used for monitoring H+ released from enzymatic reactions in real time. Since during an enzymatic reaction, only a small amount of H+ may be released, it may only cause a small pH change in the bulk solution, especially when the buffer capacity is high. The highly sensitive method of the present invention may be able to detect the small localized and temporal pH changes with a good spatial resolution. This type of LC-based detecting may find utilities in high throughput screening of potential enzyme substrates and enzyme inhibitors and may be used to monitor enzymatic reactions.

In one embodiment, the binding agent may be penicillin and the biological molecule may be penicillinase. The change in pH detected may be at least 0.1 and the pH of the sample before the contacting of the binding agent may be below 7. The hydrolysis of δ-lactam antibiotics by penicillinase releases H+ and decreases pH in the vicinity of penicillinase-modified region which may be detected by the method according to any aspect of the present invention. In particular, the method of the present invention shows high sensitivity (1 nM within 7 min) and specificity (only β-lactam antibiotics can be detected) to monitor the enzymatic reaction.

### BRIEF DESCRIPTION OF THE FIGURES

FIG. 1 (a) shows a schematic illustration of a substrate and a hydrophilic layer with embossed microfluidic channels, herein referred to as the microfluidic section (10) of an example of a detection system of the present invention.

FIG. 1 (b) shows a cross section SEM image of the microfluidic channels (20) showing detailed dimensions of the microfluidic channels (20) (W×D=200 μm×160 μm).

FIG. 2 (a) shows images of LC taken under a polarized microscope (crossed polars) with IgG as the biological molecule on the contact portion of the DMOAP coated glass slide.

FIG. 2 (b) shows images of LC taken under a polarized microscope (crossed polars) with bi-Bovine serum albumin (BSA) as the biological molecule on the contact portion of the DMOAP coated glass slide.

FIG. 2 (c) shows a schematic illustration of label-free detection mechanism based on LCs.

FIG. 3 shows the correlation between the length of the bright LC regions and the concentration of anti-IgG. (a) shows images of LC taken under a polarized microscope (crossed polars) with IgG as the biological molecule on the contact portion of the DMOAP coated glass slide and with varying concentration of anti-IgG as binding agent. The concentration of anti-IgG is (i) 0.02, (ii) 0.05 and (iii) 0.08 mg/mL, respectively; (b) shows the linear relationship between the length of the bright region and the anti-IgG concentration.

FIG. 4 shows an example of a detection system of the present invention developed in microfluidic channels for high throughput antibody detection. (a) shows a schematic illustration of the detection system used in an immunoassay; (b) shows the images of LC taken under a polarized microscope (crossed polars) which are the test results of the immunoassay. LC only appears bright in the line-line intersections where antigens as biological molecules and their respective antibodies as binding agents meet.

FIG. 5 shows a schematic configuration of a copper grid described in Example 4 impregnated with PBA-doped 5CB and exposed to penicillin G aqueous solution. The magnified features show schematic illustrations of the immobilization of penicillinase and the enzymatic reaction of penicillinase on the surface of the copper grid.

FIGS. 6(a) to (d) show optical images (crossed polars) of copper grids impregnated with (a-b) 0.3% PBA-doped 5CB and (c-d) 5CB, after they were exposed to aqueous solution at (a) and (c) with pH=7.0, (b) and (d) with pH=6.0.

FIG. 6(e) shows optical images (crossed polars) of copper grids impregnated with 0.3% PBA-doped 5CB when exposed to aqueous solutions at different pH.

FIGS. 7 (a) to (c) show optical images (crossed polars) of 5CB (doped with 0.3% of several carboxylic acid compounds) immersed in aqueous solutions of four different pH values. These compounds are (a) 4-biphenylcarboxylic acid, (b) acetic acid, and (c) lauric acid.

FIG. 8 shows the optical response of 5CB to H+ released from enzymatic reaction. Copper grids with immobilized penicillinase were immersed into PBS buffer (pH=7.0) containing different substrates. (a) 1 mM penicillin G, (b) no substrate, (d) 1 mM penicilloate, (e) 1 mM ampicillin, (f) 1 mM tetraglycine, and (g) 1 mM HCl. In some experiments, copper grids without immobilized penicillinase were used. These grids were immersed in PBS buffer containing (c) 1 mM penicillin G and (h) 1 mM HCl.

FIG. 9 (a) shows the influence of the concentrations of penicillin G on optical images (crossed polars) of 0.3% PBA-doped 5CB confined in copper grids at different time. The copper grids were coated with 0.2 mg/mL penicillinase at 4° C. for 12 h.

FIG. 9 (b) shows the increase in planar coverage when 0.3% PBA-doped 5CB confined in copper grids was exposed to different concentrations of penicillin G at different exposure times.

FIG. 10 (a) to (c) show the optical images (crossed polars) of 0.3% PBA-doped 5CB confined in a bar-shaped, penicillinase-modified grid after contacting with 20 and 100 nM penicillin G, respectively at different times.

FIGS. 11 (a) to (d) show optical images (crossed polars) of copper grids impregnated with 0.3% PBA-doped 5CB, after they were exposed to phosphate buffer solution (pH=7.0) containing (a) 100 μM penicillin G, (b) 100 μIM ampicillin, (c) 100 μM HCl, and (d) 100 μM tetraglycine. In panel (I), the copper grids were coated with 0.2 mg/mL penicillinase at 4° C. for 12 h and in panel (II), copper grids were unmodified.

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

Having now generally described the invention, the same will be more readily understood through reference to the following preferred embodiment and examples which are provided by way of illustration, and are not intended to be limiting of the present invention.

### DETAILED DESCRIPTION OF THE PREFERRED EMBODIMENT

FIG. 1 (a) shows a schematic illustration of a glass slide 14 as substrate and poly (dimethylsiloxane) (PDMS) layer 12 as a hydrophilic layer with embossed microfluidic channels 20, herein referred to as the microfluidic section 10. This microfluidic section 10 comprises three channels 20a, 20b and 20c supported on a DMOAP coated glass slide 14 and sealed with poly (dimethylsiloxane) (PDMS) 12 with embedded microfluidic channels 20. Each microfluidic channel 20a, 20b and 20c begins with an inlet reservoir 16 for pipetting of a sample and/or a binding agent to enter the microfluidic channels 20 by capillary action and to be deposited on the contact portion of the glass slide 14. Each microfluidic channel 20a, 20b and 20c ends with an outlet reservoir for collection of excess sample and/or a binding agent that does is not deposited on the contact portion of the glass slide 14.

The DMOAP-coated glass slide 14 was prepared with the established method as reported (Xue et al, 2008), herein incorporated by reference.

The surface of PDMS layer 12 with embossed microfluidic channels 20 was prepared by casting Sylgard 184 on a silicon master with raised features (W×D×L:200 μm×160 μm×50.9 mm). The silicon master was fabricated by defining the channel patterns via photolithography onto negative photoresist (SU8-2050, Microchem, MA) spun coated onto a silicon wafer. PDMS was then degassed in vacuum to remove air bubbles, and cured at 70° C. for 3 h. After curing, the PDMS layer 12 with microfluidic channels 20 was then cut and peeled off from the master. Inlet and outlet reservoirs 16 and 18 respectively (3 mm in diameter) at both ends of microfluidic channels 20 were prepared by using a 3-mm-diameter hole puncher. Before use, the surface of the PDMS layer 12 with microfluidic channels 20 was modified by using oxygen plasma (100W, 45 s) to increase its hydrophilicity. Finally, a closed microfluidic portion 10 was formed by binding the oxygen-plasma-treated PDMS layer 12 and a N,N-dimethyl-N-octadecyl-3-aminopropyltrimethoxysilyl chloride (DMOAP)-coated glass slide 14 together. To ensure good sealing of microfluidic channels 20, pressure was applied to the microfluidic portion 10 for 20-30 s.

FIG. 1 (b) shows a cross section SEM image of the microfluidic section (20) showing detailed dimensions of the microfluidic channels (12) (W×D=200 μm×160 μm).

The LC cell (figure not shown) can be fabricated by pairing a DMOAP-coated glass slide 14 prepared as described above with another glass slide. These two slides were aligned (facing each other) and separated from each other with a fixed distance (˜6 μm) by using two strips of Mylar films at both ends of the glass slides. The LC cell was secured with two binder clips. To fill up the empty cell with LCs, a drop of LCs 5CB was dispensed onto the edge of the cell, and the 5CB was withdrawn to the space between two glass slides by capillary force. Finally, the optical textures of the LCs inside the optical cell were observed under crossed polars with a polarized microscope (Nikon ECLIPSE LV100POL, Tokyo, Japan) in the transmission mode.

In use, the surface of the DMOAP coated glass slide 14 is coated with a layer of antigen (IgG or bi-BSA). Buffer solutions containing different antibodies (without labelling) can be pipetted into the inlet reservoirs 16 (indicated by arrows), allowing them to enter the microfluidic channels 20 by capillary action. After incubation and rinsing, a thin layer of LCs is then supported on the DMOAP coated glass slide 14 to report the result of the immunoassay.

### EXAMPLES

Standard molecular biology techniques known in the art and not specifically described were generally followed as described in Sambrook and Russel, Molecular Cloning: A Laboratory Manual, Cold Springs Harbor Laboratory, New York (2001).

### Example 1

Stock solutions, proteins IgG, anti-IgG, bi-Bovine serum albumin (BSA), anti-biotin and BSA were dissolved in 0.1 M phosphate-buffered saline (PBS, pH 7.4). Prior to use, stock solutions were diluted with PBS buffer to obtain desired concentrations.

To show that LCs supported on DMOAP-coated glass slides can be used to prepare quantitative microfluidic immunoassays solution containing 0.003 mg/mL of IgG was applied to the entire surface of a DMOAP-coated glass slide 14. DMOAP-coated glass slide 14 is known to adsorb proteins strongly and align LCs homeotropically (perpendicular to the surface). After incubation for 2 h at room temperature, the IgG decorated DMOAP-coated glass slide 14 was rinsed with PBS buffer and dried under a stream of nitrogen. 20 μL drop of anti-IgG was pipetted into first inlet reservoir 16 which subsequently entered the microfluidic channel 20a by capillary action over the IgG-decorated DMOAP-coated glass slide 14. 20 μL drop of anti-biotin was pipetted into second inlet reservoir 16 which subsequently entered the microfluidic channel 20b by capillary action over the IgG-decorated DMOAP-coated glass slide 14. After 30 min, the PDMS layer 12 was removed and the IgG-decorated DMOAP-coated glass slide 14 was rinsed with PBS with 1% SDS solution and deionized water and then dried under a stream of nitrogen for subsequent analysis.

Similarly, the method disclosed above was repeated on a second DMOAP-coated glass slide 14 decorated with bi-BSA.

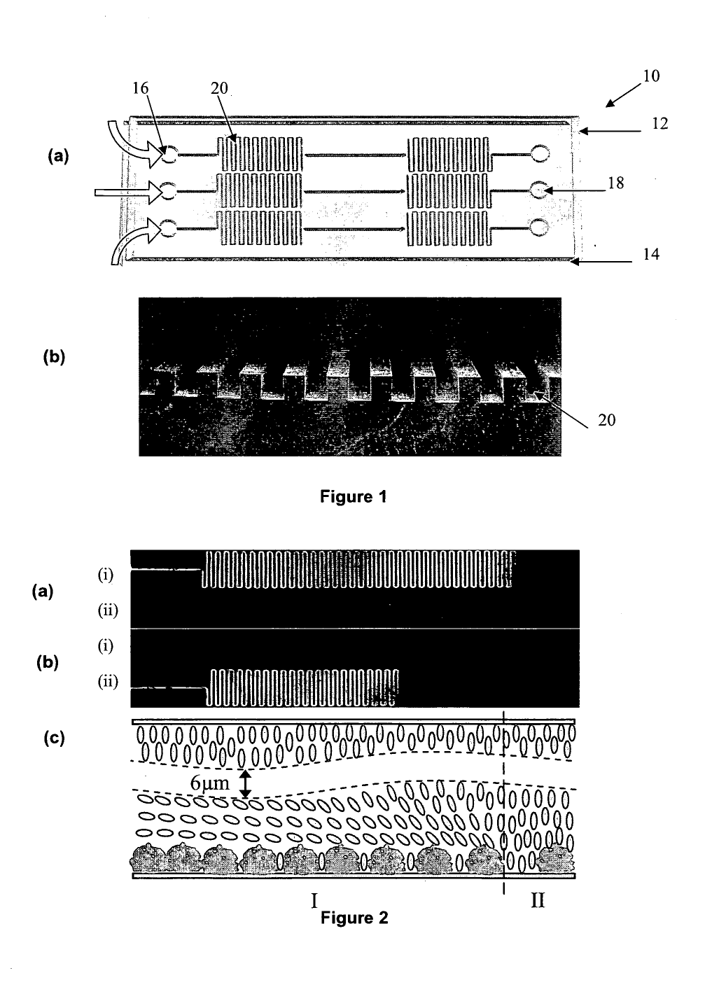

The results are shown in FIGS. 2(a) and (b). The concentration of IgG and bi-BSA used was 0.003 mg/mL, which is just below the critical concentration as disclosed in (Xue et al, 2008). Therefore, LCs supported on the IgG and bi-BSA-laden surfaces appeared dark (results not shown). Addition of 0.05 mg/mL of anti-IgG and anti-biotin solutions (both without fluorescence labeling) into microfluidic channels 20a and 20b supported on the IgG-decorated DMOAP-coated glass slide 14 followed by 30 min of incubation and rinsing with PBS buffer was expected to result in the anti-IgG binding to the IgG decorated DMOAP-coated glass slide. The surface protein concentration was expected to exceed the critical level and therefore, trigger an orientation transition of LCs. FIG. 2a shows the image of a thin layer (˜6 μm) of LCs of taken under a polarized microscope (crossed polars). The top panel (i) shows that bright colors (shown as different shades of grey) can be readily observed in the anti-IgG channel 20a, but the anti-biotin channel 20b remained dark (ii). This result shows that LCs can be used to report the binding of anti-IgG to IgG as bright optical signals, providing that the binding event causes the surface protein concentration exceeding the critical value.

The result for the second bi-BSA decorated DMOAP-coated glass slide 14 is shown in FIG. 2(b). Similarly, the top panel (i) anti-IgG channel 20a remained dark as anti-IgG did not bind with the BSA on the DMOAP-coated glass slide 14; (ii) shows that bright colors (shown as different shades of grey) can be readily observed in the anti-biotin channel 20b.

This result clearly indicates that the principle of using LCs to report immunobinding events is general enough such that it can also be used to image the binding of anti-biotin to bi-BSA.

FIG. 2 (c) shows a schematic illustration of label-free detection mechanism based on LCs.

### Example 2

To further investigate the correlation between the length of the bright LC region and the concentration of anti-IgG, used in this example as a representative antibody, different concentrations of anti-IgG solutions were pipetted into different microfluidic channels 20a, 20b and 20c supported on IgG decorated DMOAP-coated glass slide 14.

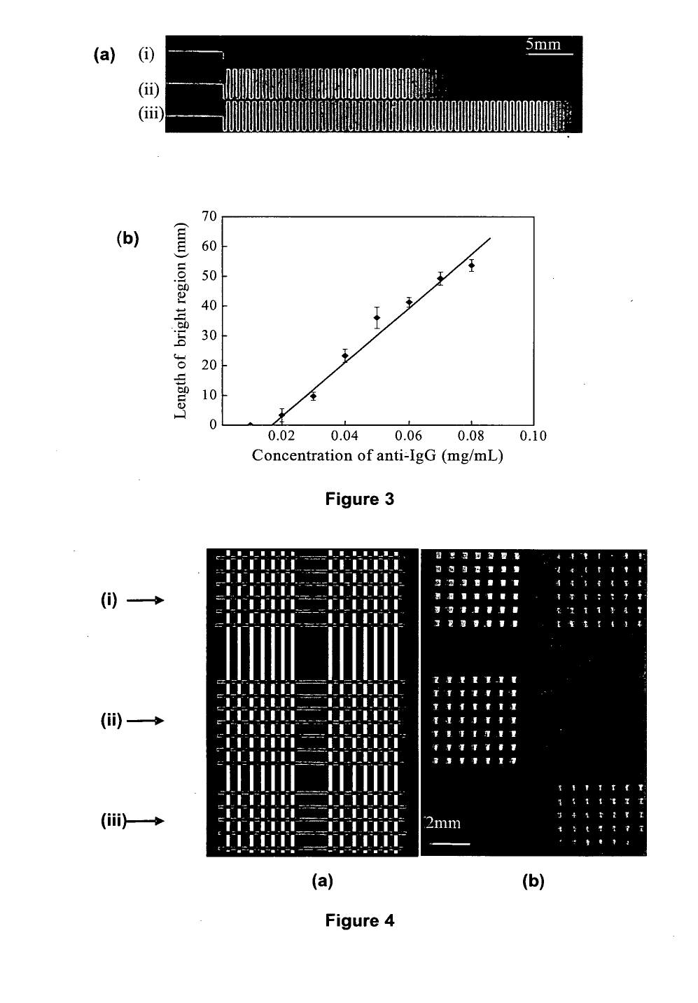

FIG. 3(a) shows an image of LC taken under a polarized microscope (crossed polars) with IgG decorated DMOAP coated glass slide 14 and with varying concentration of anti-IgG. The concentrations of anti-IgG are (i) 0.02 mg/mL, (ii) 0.05 mg/mL and (iii) 0.08 mg/mL, respectively. The results show that the lengths of the bright regions depend on the concentration of anti-IgG. When the anti-IgG concentration is below 0.02 mg/ml, the LC image remained dark. Thus, 0.02 mg/ml is the detection limit for this LC-based immunoassay. When the anti-IgG concentration is above 0.02 mg/ml the length of the bright LC region increases with the increasing of anti-IgG concentration as shown in FIG. 4(b). We may conclude from FIG. 4(b) that the length of bright LC region is proportional to the anti-IgG concentration. The least square-fitting line has a correlation coefficient of R=0.987. This indicates that the antibody concentration can be determined simply by measuring the length of the bright LC region in the microfluidic channels 20.

### Example 3

IgG and bi-BSA were first immobilized on a DMOAP coated glass slide 14 by injecting 104 of both solutions (0.003 mg/mL) through microfluidic channels 20 as shown in FIG. 4a (from bottom to top). After 20 min of incubation, the first PDMS layer 12 with microfluidic channels 20 was peeled off and the IgG and bi-BSA decorated DMOAP coated glass slide 14 was rinsed with buffer solutions and dried with nitrogen. Subsequently, 10 μL of solutions of anti-IgG (i), anti-biotin (ii) and mixtures containing 1:1 anti-IgG and anti-biotin (iii) were injected into individual microfluidic channels 20, which run perpendicularly to the previous linear protein patterns, over DMOAP coated glass slide 14. After 20 min of incubation, the PDMS layer 14 with microfluidic channels 20 was peeled off and the IgG and bi-BSA decorated DMOAP coated glass slide 14 with antibodies were rinsed, blown dry and analyzed with LCs.

To demonstrate the feasibility of using the LC-based immunoassay for multiplex and high throughput diagnostic applications, we patterned the surface with both FIG. 4(b) shows the images of LC taken under a polarized microscope (crossed polars) which are the test results of the immunoassay. LC only appears bright (shown as white spots) in the line-line intersections where antigens and their respective antibodies meet. This suggests that the specific binding of both antigen/antibody pairs can be detected with LCs in a multiplexed manner. Moreover, since the mixture contains both anti-IgG and anti-biotin, line-line intersections between mixture/IgG and mixture/bi-BSA both appear bright. Finally, we notice that the interference colour is yellow (cannot be seen) in the biotin/anti-biotin intersection whereas the interference colour in the IgG/anti-IgG intersection is white. The difference in the interference colour (yellow vs white) shows that more anti-biotin can bind to the biotin surface due to a faster binding rate.

### Example 4

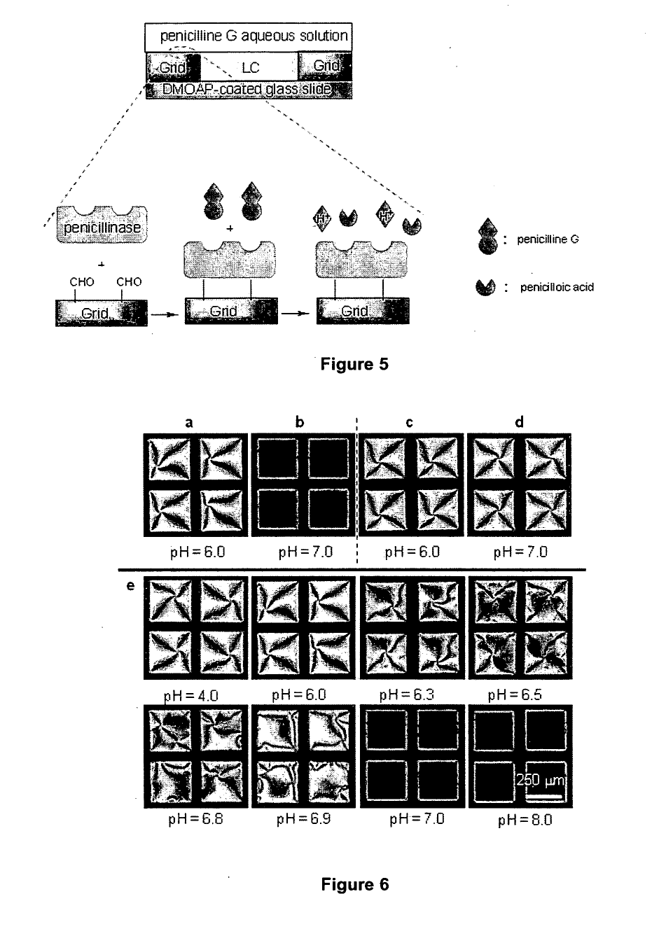

An LC-based sensor for real-time monitoring of changes in local pH values near a solid surface and its application for monitoring activities of enzymes immobilized on the surface. The hydrolysis of penicillin G by immobilized penicillinase was monitored using the method even when the concentration of penicillin G was as low as 1 nM. A schematic illustration of the copper grid impregnated with PBA-doped 5CB and exposed to penicillin G aqueous solution is shown in FIG. 5.

### Immobilization of Penicillinase on Copper Grids

TEM copper grids (75 mesh, Electron Microscopy Sciences, U.S.A.) were cleaned in methanol, ethanol, and acetone (sonication for 15 min in each solvent), and heated overnight at 100° C. to evaporate residual solvents. The clean grids were immersed in an aqueous solution containing 5% of polyethylenimine (PEI). After 30 min, the grids were washed thoroughly with deionized water and dried in a 100° C. oven. The grids were then immersed in an aqueous solution containing 5 wt % of glutaraldehyde and 10 mM of sodium cyanoborohydride for 2 h. Finally, the grids were incubated in a buffer (which contained 0.5 mM sodium phosphate and 50 mM sodium chloride and was degassed for at least 1 h before use) containing 0.2 mg/mL of penicillinase for 12 h at 4° C. For the bar-shaped grid, 0.5 μL of buffer solution containing penicillinase was dispensed onto one side of the grid. To prevent the evaporation of the penicillinase solutions, the grid was stored in a sealed and humid chamber for 12 h at 4° C. Excess penicillinase was removed by incubating the grid in 2×SSPE buffer (300 mM NaCl, 23 mM NaH₂PO₄, 2.8 mM EDTA) and 1% Triton X-100 for 10 min. All penicillinase-modified grids were kept at 4° C. before use.

### Preparation of LC Samples

Cleaned glass slides were immersed in 0.1 wt % DMOAP solution to obtain a layer of DMOAP on the surface. The glass slides were then cut into small squares (5 mm×5 mm) and used as substrates for supporting LC. For fabrication of LC based pH sensor, an unmodified copper grid was placed on the DMOAP-coated glass slide. Then, approximately 0.3 μL of 0.3 wt % PBA-doped 5CB was dispensed onto the grid, and excess LC was removed by using a capillary tube. The grid containing LC was covered with 300 μL of buffer solutions of different pH values. For monitoring enzymatic activities, the unmodified copper grid was replaced by a penicillinase-modified copper grid. After filling the grid with PBA-doped 5CB, the grid was immersed in 300 μL of sodium phosphate buffer (pH=7.0) with different concentrations of penicillin G. The optical appearances and fluorescence images of these samples were observed by using polarizing optical microscope and fluorescence microscope, respectively.

FIG. 6 shows optical images (crossed polars) of copper grids impregnated with (a-b) 0.3% PBA-doped 5CB and (c-d) 5CB, after they were exposed to aqueous solution at (a) and (c) pH=7.0, (b) and (d) pH=6.0. As can be seen the orientation of LC changes with different pH. FIG. 6(e) shows the orientational transition of 5CB because of the local pH change. FIG. 6(f) shows the optical images (crossed polars) of copper grids impregnated with 0.3% PBA-doped 5CB when they were exposed to aqueous solutions at different pH. As can be seen, the orientation of PBA-doped 5CB changes with different pH.

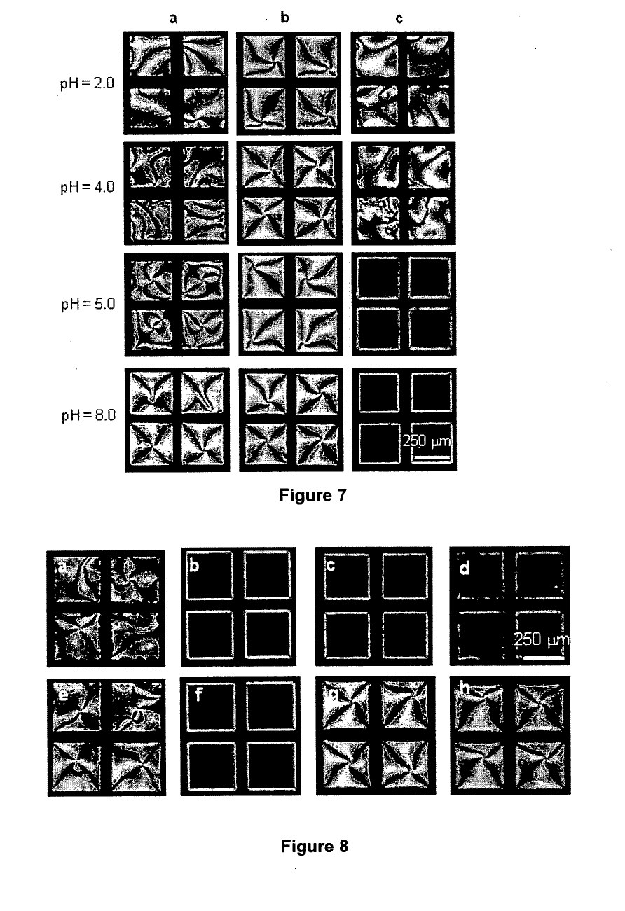

Similarly, FIG. 7 shows optical images (crossed polars) of 5CB (doped with 0.3% of several carboxylic acid compounds) immersed in aqueous solutions of four different pH values. These compounds are (a) 4-biphenylcarboxylic acid, (b) acetic acid, and (c) lauric acid. As can be seen, they are not as effective in detecting changes in pH and even then they are only able to detect large changes in pH.

Detection of H⁺ released from enzymatic reaction of penicillinase was measured. As can be seen in Panel I of FIG. 8, copper grids were coated with 0.2 mg/mL penicillinase at 4° C. for 12 h and then exposed to: (a) 1 mM penicillin G, and (b) pure sodium phosphate buffer as negative control. (c) An unmodified copper grid was exposed to 1 mM penicillin G and used as a control. As can been seen, the change in orientation of PBA-doped 5CB only took place in (a) and not in B where no H+ was released as no enzymatic reaction took place between penicillinase and sodium phosphate buffer.

In panel II of FIG. 8, the results of the specificity of the PBA-doped 5CB pH sensor is shown. Copper grids were coated with 0.2 mg/mL penicillinase at 4° C. for 12 h and then exposed to: (d) 1 mM ampicillin, (e) 1 mM tetraglycine, and (f) 1 mM HCl. (g) An unmodified copper grid was exposed to 1 mM HCl as positive control. All of the substrates were dissolved in sodium phosphate buffer (pH=7.0). As can be seen in the results, the orientation of the PBA-doped 5CB changes in (d), (f) and (g) as the pH changes.

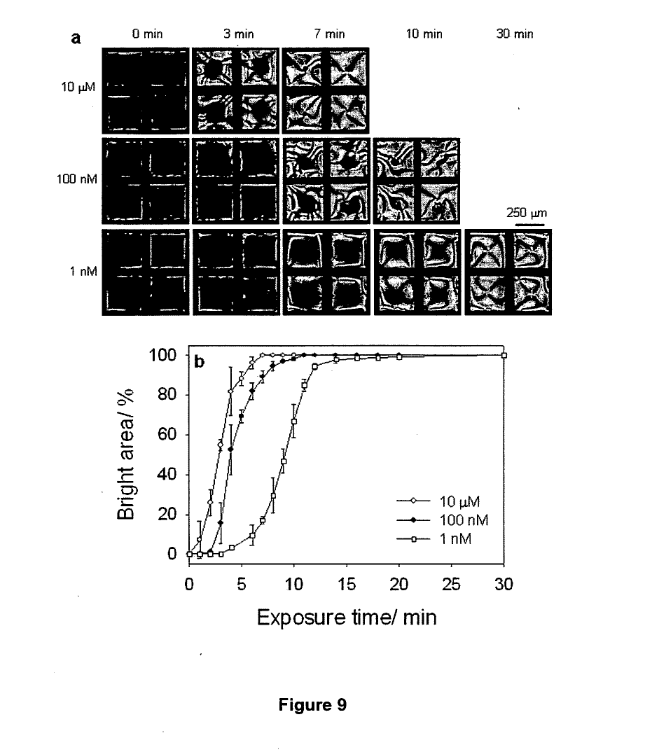

The influence of the concentrations of penicillin G on optical images (crossed polars) of 0.3% PBA-doped 5CB confined in copper grids at different times is shown in FIG. 9 (a). The copper grids were coated with 0.2 mg/mL penicillinase at 4° C. for 12 h. As can be seen the presence of penicillin G can be detected at a low concentration of 1 nM in 7 mins as the orientation of the PBA-doped 5CB changes.

FIG. 9(b) shows the increase in planar coverage when 0.3% PBA-doped 5CB confined in copper grids was exposed to different concentrations of penicillin G at different exposure times.

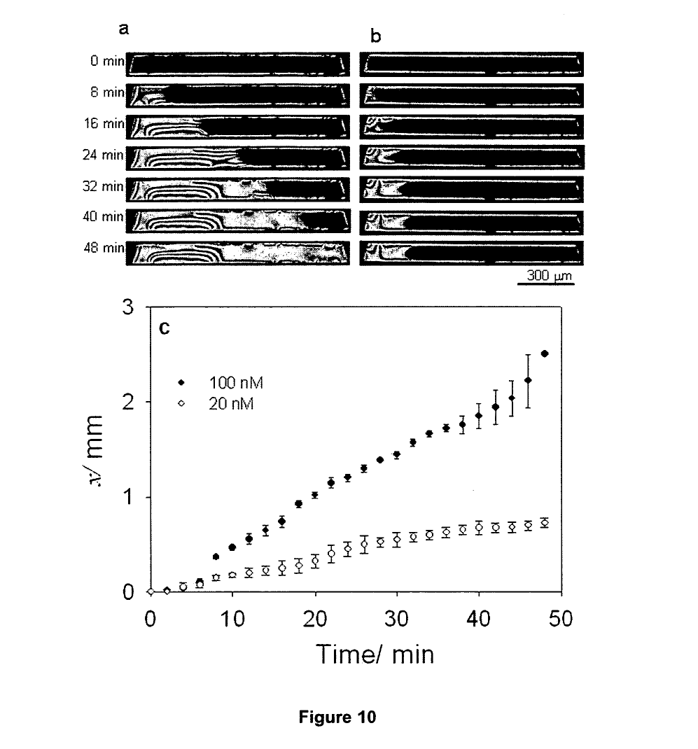

Similarly, FIG. 10 shows optical images (crossed polars) of 0.3% PBA-doped 5CB confined in a bar-shaped, penicillinase-modified grid after contacting with 20 and 100 nM penicillin G, respectively at different time.

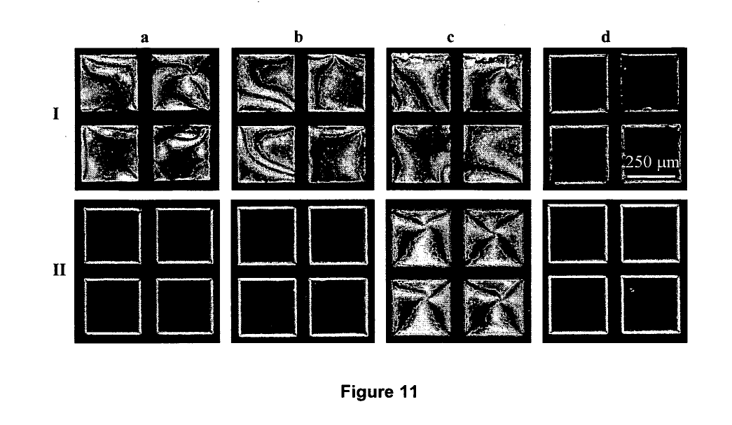

After the copper grids were modified with penicillinase, approximately 0.1 μL of 0.3 wt % PBA-doped 5CB was dispensed onto the grid. Then, the grid was put onto the surface of sodium phosphate buffer containing different analytes. The results are shown in FIG. 11. In panel I, the copper grids were coated with 0.2 mg/mL penicillinase at 4° C. for 12 h and then in (a) 100 μM penicillin G, (b) 100 μM ampicillin, (c) 100 μM HCl, and (d) 100 μM tetraglycine. Panel II shows the results with unmodified copper grids.

### REFERENCES

1. C.-Y. Xue, K.-L. Yang, Langmuir (2008), 24, 563.
2. Sambrook and Russel, Molecular Cloning: A Laboratory Manual, Cold Springs Harbor Laboratory, New York (2001).

1. A method of detecting and/or quantifying the presence of at least one biological molecule in at least one sample, the method comprising the steps of:
(a) contacting a substrate with the at least one sample at a contact portion of said substrate;
(b) providing at least one binding agent specific to the biological molecule on said contact portion;
(c) disposing a liquid crystal at said contact portion with the sample and binding agent;
(d) determining whether the orientation of the liquid crystal changes after the sample contacts the binding agent, indicating the presence of the biological molecule; and
(e) determining length of bright region of the liquid crystal and/or change in interference colour of said liquid crystal and consequently, indicating the quantity of said biological molecule;
wherein the sample and/or binding agent are provided to the substrate by way of at least one microfluidic channel.

2. The method according to claim 1, wherein step (b) is carried out before step (a).

3. The method of claim 1, wherein the sample contacts the substrate by way of microfluidic channels running perpendicular to microfluidic channels providing the binding agent specific to the biological molecule on to the substrate.

4. The method according to claim 1, further comprising a step of removing the microfluidic channel from the substrate after steps (a) and (b).

5. The method according to claim 1, wherein the microfluidic channels are formed within at least one hydrophilic surface.

6. The method according claim 5, wherein the hydrophilic surface is made from Polydimethylsiloxane (PDMS) and/or Poly(methyl methacrylate) (PMMA).

7. The method according to claim 1, wherein the substrate is made from any one or a combination of polymer, ceramic, glass, metal, composite thereof and/or laminate thereof.

8. The method according to claim 1, wherein the substrate is coated with N,N-dimethyl-n-octadecyl-3-aminopropyltrimethoxysilyl chloride (DMOAP).

9. The method according to claim 1, wherein the biological molecule is at least one of: DNA, RNA, protein, virus, bacteria, pathogen, antigen, or antibody.

10. The method according to claim 1, wherein the binding agent is at least one antibody.

11. The method according to claim 1, wherein the liquid crystal is a lyotropic liquid crystal

12. The method according to claim 1, wherein the contacting of the biological molecule and the binding agent results in change in at least one parameter, the change detectable and/or quantifiable by the liquid crystal.

13. The method according to claim 12, wherein the parameter is at least temperature, pH, concentration of H+ or concentration of at least one enzyme substrate.

14. The method according to 1, wherein the liquid crystal is at least one 4′-pentyl-biphenyl-4-R, wherein R is at least one functional group selected from carboxylic acid, amine, aldehyde, and oligopeptide.

15. The method according to claim 14, wherein the 4′-pentyl-biphenyl-4-R is 4-cyano-4′-pentylbiphenyl (5CB) or poly(p-benzamide (PBA) doped 5CB.

16. An in vitro method for diagnosing a subject as having a disorder, wherein the method is according to claim 1, wherein the presence of the biological molecule indicates the presence of the disorder.

17. The in vitro method according to claim 16, wherein the disorder is Dengue fever, AIDS, Sexually Transmitted Disease, Hepatitis, or antibiotic resistance.

18. A detection system for carrying out a method according to claim 1, the detection system comprising:
a substrate and a hydrophilic layer with embossed microfluidic channels, engageable to form a path to transmit at least one sample and/or binding agent to the substrate via the microfluidic channels;
wherein the substrate is removable to form a component of a liquid crystal cell for optical detection of bound biological molecule and binding agent.

19. The detection system according to claim 18, wherein the liquid crystal cell comprises the removable substrate and a transparent cover over the substrate.

20. The detection system according to claim 19, wherein the transparent cover is a glass slide.

21. The detection system according to claim 18, for use in quantitative protein detection.

22. A method of quantifying and/or detecting the presence of at least one biological molecule in at least one sample, the method comprising the step of determining length of bright region of liquid crystal in contact with said sample and/or change in interference colour of said liquid crystal in contact with said sample, consequently indicating the quantity of said biological molecule in said sample.

23. A detection system for detecting the presence of at least one biological molecule in at least one sample, the detection system comprising:
a substrate and a hydrophilic layer with embossed microfluidic channels, engageable to form a path to transmit the at least one sample and/or binding agent to the substrate via the microfluidic channels;
a liquid crystal cell mounting for receiving the substrate to form a liquid crystal cell;
wherein the substrate is removable from the hydrophilic layer and mountable to the liquid crystal cell mounting for optical detection of bound biological molecule and binding agent.

24. A method of detecting a biological molecule in at least one sample using at least one 4′-pentyl-biphenyl-4-R, wherein R is at least one functional group selected from carboxylic acid, amine, aldehyde, and oligopeptide, and the 4′-pentyl-biphenyl-4-R is capable of detecting at least one change in at least one parameter resulting from contacting at least one binding agent specific to the biological molecule with the sample.

25. The method according to claim 24, wherein the parameter is at least temperature, pH, concentration of H+ or concentration of at least one solvent molecule.

26. The method according to claim 24, wherein the biological molecule is at least one enzyme.

27. The method according to claim 24, wherein the binding agent is at least one β-lactam antibiotic.

28. The method according to claim 27, wherein the β-lactam antibiotic is penicillin and the biological molecule is penicillinase.

29. The method according to claim 1, wherein the parameter is a change in pH and the 4′-pentyl-biphenyl-4-R is poly(p-benzamide (PBA) doped 5CB.

30. The method according to claim 29, wherein the change in pH is at least 0.1.

31. The method according to claim 24, wherein the pH of the sample before the contacting of the binding agent is below 7.
